(19) United States
(12) Patent Application Publication
(10) Pub. No.: US 2006/0200094 A1
(43) Pub. Date: Sep. 7, 2006

(54) System for metering and delivering a liquid medium, especially for enteral feeding

(76) Inventor: Christian Holz, Eitorf (DE)

Correspondence Address: EDWARDS & ANGELL, LLP, P.O. BOX 55874, BOSTON, MA, 02205, US

(21) Appl. No.: 10/559,843

(22) PCT Filed: Jun. 16, 2004
(86) PCT No.: PCT/EP04/06495

§ 371 Date: Dec. 7, 2005

(30) Foreign Application Priority Data
Jun. 18, 2003 (DE) 203 09 513.8

(51) Int. Cl. A61M 5/14; A61M 5/00

(52) U.S. Cl. 604/251

(57) ABSTRACT

A system (1) for metering and delivering a liquid medium, in particular for enteral nutrition in medical applications, comprises a storage container (3) having a certain volumetric capacity and a supply device (4) and a discharge device (5) for the medium, whereby the supply and discharge of the medium to and from the storage container (3) is effected by the force of gravity. A detecting device, e.g. in the form of a pair of spaced diode measuring devices (6, 7) serves for determining at least a lower and at least an upper filling level (8, 9) of the medium in the storage container (3) and for delivering appropriate detection signals. Controllable actuating organs (10, 11) serve for closing or opening the supply device (4) and the discharge device (5), whereby the detection signals from the detecting device (6, 7) are applicable to an internal or external control unit in order to deliver setting signals to the controllable actuating organs (10, 11) according to a given program sequence in dependence on the detection signals. The system enables enteral nutrients to be supplied to a patient with a precise volumetric dosage. It does not need a feed pump.

The invention relates to a system for metering and delivering a liquid medium, in particular for enteral nutrition in medical applications, in accordance with the preamble of claim 1.

A system of the type forming in question which is based on the force of gravity is known from EP 0 241 595 A1 for example. In this system, a container aligned in the direction of the gravitational force serves for storing a quantity of nutriment. The container is connected by a plug-in tube and an associated inlet hose to a drip chamber which cooperates with a hose clip through an outlet hose extending to a patient. The supply of the nutriment to the patient is effected continuously and can be metered out by means of the hose clip by observing the quantity of nutriment dripping into the drip chamber over a period of time and raising or lowering the amount by changing the cross section of the passage in the inlet hose by means of the hose clip. Gravimetric systems of this type differ fundamentally from other systems that are likewise used for enteral nutrition (DE-A-2855270, EP-B-923394) in that no feed pump is necessary. On the other hand, the volumetric flow of the known nourishment system is determined by the number of drops per unit of time, whereby the droplet size can be affected by many external and internal factors. This has the disadvantage that the volumetric flow rate can only be set extremely inaccurately and hence administration of a precise quantity of nutriment is not possible. Moreover, experienced trained personnel are always required in order to set the number of drops per unit of time to an approximately sufficient level of accuracy.

An object of the invention is to provide a gravity feed system of the type indicated in the preamble of claim 1 which enables precise volumetric dosage and supply of a liquid medium to a consumer, in particular for medical applications in the field of enteral nutrition.

This object is achieved by the combination of features indicated in claim 1. The invention envisages the arrangement of a detecting device for determining at least a lower and at least an upper filling level in a storage container so that one is no longer dependent on the number and size of the drops per unit of time for the precise setting of the volume that is to be applied. Both can be affected by external and internal factors which do not come into play in this invention. It can be ensured by means of controllable actuating organs for closing and opening the supply device and the discharge device that only the volume prescribed for the application will actually be supplied to a consumer or a patient in a temporal sequence defined by a control unit. At the same time thereby, the outflow of the medium volume that is to be applied from the storage container can be monitored so that an appropriate indication of malfunctioning will be perceptible in the event of a cessation of the flow of the medium per defined unit of time.

Preferably, the detecting device comprises at least two diode measuring units which detect the lower and upper filling level in the storage container. Diode measuring units work very precisely and are utilisable in the most varied of arrangements.

In order to prevent falsification of the results of the measurements made by the diode measuring units in a further embodiment of the invention, at least the upper diode measuring unit in the direction of the gravitational force is arranged in such a manner that it does not scan the stream of medium flowing into the storage container. A measurement in the incoming stream of medium could lead to premature closure of the supply device for the storage container even though the volume intended for the application has not yet entered the storage container.

Although other arrangements of the means for actuating the controllable organs could be used, these preferably each comprise a bi-stable solenoid, a continuously powered solenoid or a stepping motor which moves the respective controllable actuating organ into its closed or open position. Solenoids or stepping motors are characterised by their short response times so that the respective processes of closing or opening the supply and/or discharge device initiated by the control unit are adapted to be carried out rapidly. Owing to the fast response time, the precision of the system can be further optimised.

Since high standards of safety are demanded in the field of dosage and supply systems for enteral nutrition, detecting means are preferably provided for detecting the position of the controllable organs. Due to this position detecting function, a malfunction of the controllable organs can be immediately established and the system be turned off in good time before any possible misapplications.

Finally, the storage container in the system in accordance with the present invention is provided with a ventilating device so that there can be no falsification of the volume of medium flowing into or out of the storage container due to the presence of air.

The invention will be described in more detail hereinafter with the aid of an embodiment and reference to the drawing. In the drawing:

FIG. 1 is a schematic view of a system according to the invention with a housing shown in the opened state;

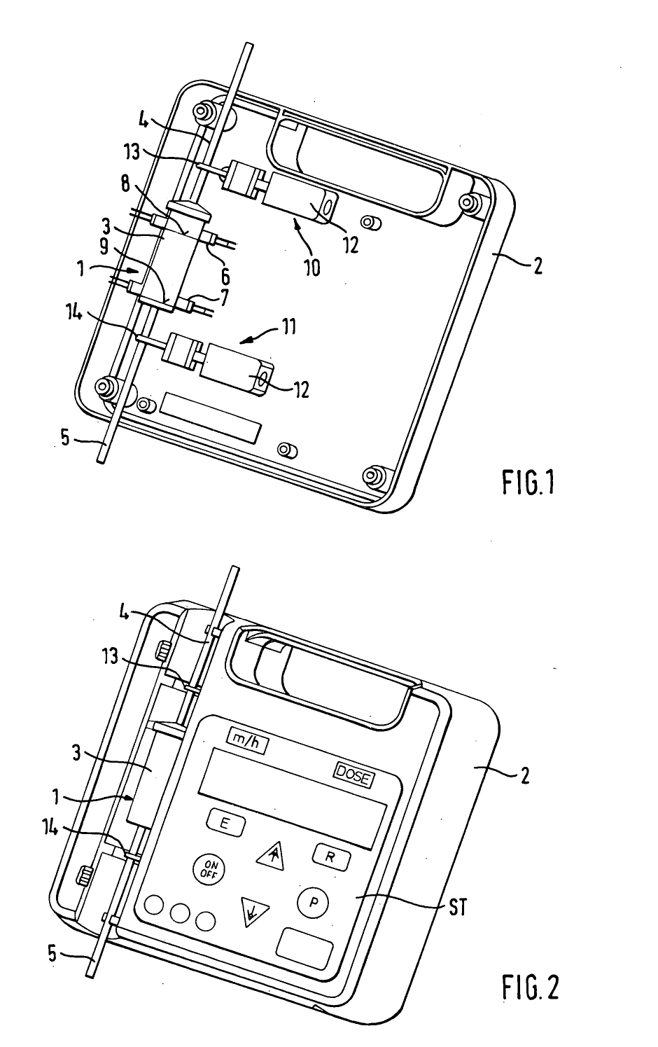

FIGS. 2 to 6 are views similar to FIG. 1 with the housing closed for illustrating the different operational states of the system from the filling of a storage container up to the supply of the medium in the storage container to a consumer; and

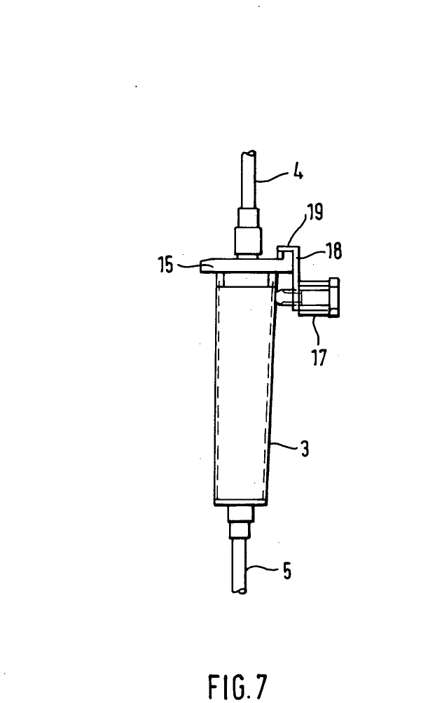

FIG. 7 is a schematic view of an embodiment of a storage container with a ventilating device.

The system in accordance with the invention bears the general reference symbol 1 in FIG. 1. The system 1 is accommodated in a housing 2 which can contain a control unit ST which will be discussed later, c.f. FIG. 2.

The system 1 comprises a storage container 3 having a supply device 4 and a discharge device 5 to which respective supply and discharge hoses can be attached as shown in FIG. 1. Both the supply device 4 and the discharge device 5 are formed in one-piece with the storage container 3, although they could also be separate components for connection to the storage container.

Furthermore, a detecting device is provided in the housing 2 for detecting a lower or minimum and an upper or maximum filling level which are respectively indicated by 8 and 9 in FIG. 1. The detecting device is connected to the control unit ST which can be constructed in a manner known to the skilled person. Apart from a programmable computer member, the control unit ST may comprise, in particular, a control panel with operating elements and a display field. The detecting device preferably comprises a pair of diode measuring units 6, 7 spaced from each other in the direction of the gravitational force. Other suitable measuring units could likewise be used. The power supply for the system 1 can be effected from an external source of energy or internally by means of a battery.

The diode measuring unit 6 for detecting the upper filling level 8 is positioned in such a way that it can scan the upper filling level 8, while it is prevented from scanning an incoming stream of medium in order to prevent the scanning results from being falsified. As an alternative to the diode measuring units 6, 7 that are arranged laterally of the storage container 3, suitable level measuring units could also be arranged on the lower and/or upper side of the storage container 3.

Furthermore, controllable actuating organs 10 and 11 are provided in the housing 2, each of said organs comprising an actuator 12 which may be in the form of a bi-stable solenoid, a continuously powered solenoid or a stepping motor.

Opening and closing elements in the form of hose squeezing elements 13, 14 are provided and these are movable into a closed or an open position by the actuating organs 10 and 11 associated therewith in order to position the supply and discharge devices 4, 5. The squeezing elements 13, 14 are in the closed position in FIG. 1. Other suitable closing elements could also be provided, e.g. closure valves in place of the hose squeezing elements.

The actuation of the upper and lower squeezing elements 13, 14 together with the associated controllable actuating organs 10, 11 can be controlled by the control unit ST which receives as an input the detection signals from the upper and lower diode measuring units 6, 7 and then processes these signals according to a desired program sequence. Accordingly, different control sequences could be used in dependence on the programming of the control unit.

The control unit ST can be integrated into the system, or it may be an external control unit. In the latter case, an interface can be provided in the system in order to exchange the signals between the control unit and the signal generators and receivers unique to the system.

The mode of functioning of the system 1 according to the invention is described hereinafter with reference to FIGS. 2 to 6.

In FIG. 2, the system 1 is in its operational state and the storage container 3 may be filled or empty. In correspondence with FIG. 2, the storage container 3 is to be aligned in such a way that the force of gravity can be maximally effective. The two squeezing elements 13, 14 are in the closed position. This state is also referred to as the secured state because the medium cannot be supplied to a consumer due to the closed position of the two squeezing elements 13, 14. For the purposes of this secured state, it would also be sufficient if only the lower squeezing element 14 were to be in the closed position.

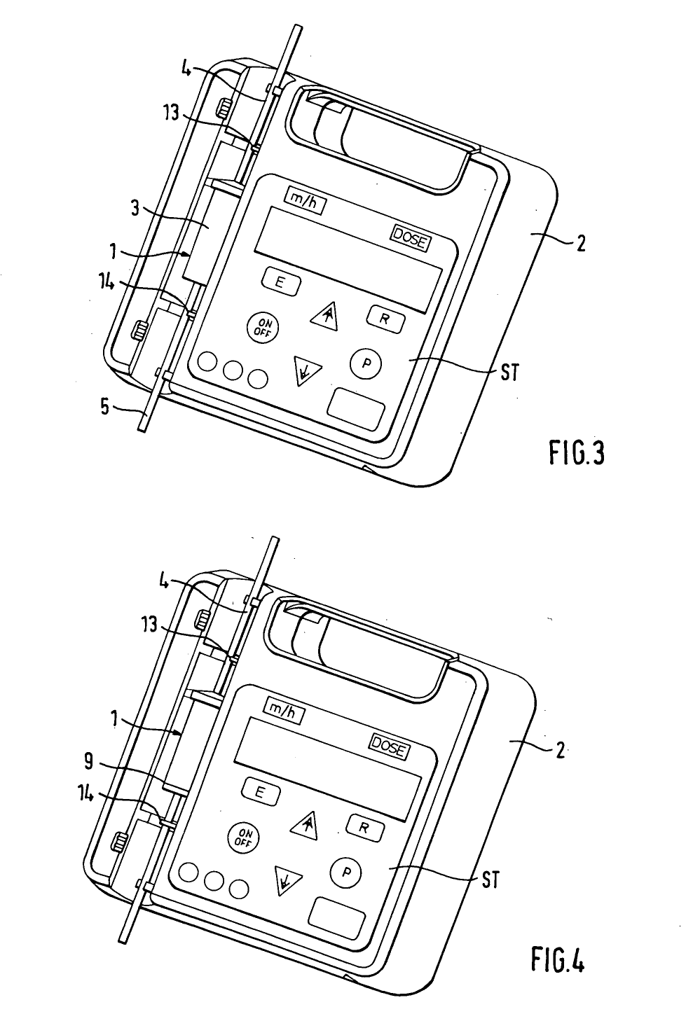

In contrast to the secured state in accordance with FIG. 2, the system 1 is illustrated in FIG. 3 in a replacement state wherein the two squeezing elements 13, 14 are likewise in the open position. In this state, replacement of the storage container 3 can take place, this having to be effected e.g. once per day when the system 1 is being used for enteral nutrition.

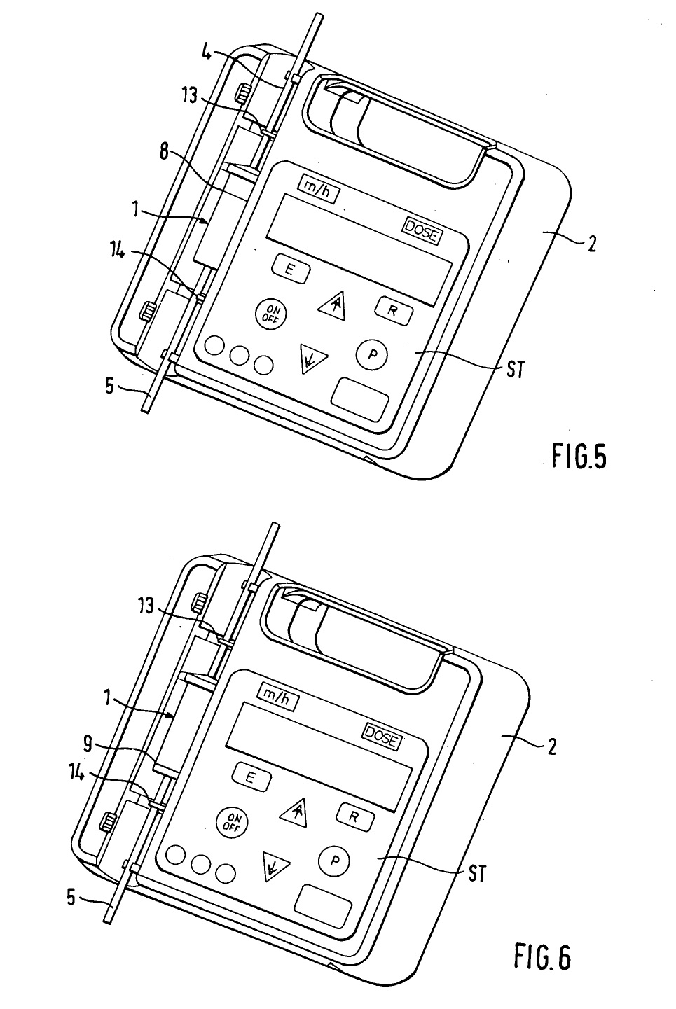

In FIG. 4, the system 1 is shown in the state wherein the storage container 13 is being filled. Here, the upper squeezing element 13 is in its open position, whereas the lower squeezing element 14 is in the closed position. When a medium flows through the supply device 4 into the storage container 3, the lower diode measuring unit 7 firstly detects the lower filling level 9 and supplies a signal to this effect to the control unit. The inflowing medium continues to fill the storage container 3.

In accordancee with FIG. 5, the flow of the medium into the storage container 3 through the supply device 4 continues until the medium has reached the upper filling level 8. Upon reaching the upper filling level 8, an appropriate detection signal is supplied to the control unit ST by the upper diode measuring unit and as a result thereof the upper squeezing element 13 is moved from the open position into the closed state thereof so that no further inflow of the medium will take place.

After closure of the upper squeezing element 13, the lower squeezing element 14 is opened by controlling the lower actuating organ 11 by means of an operating signal supplied by the control unit ST so that the volume of medium contained in the storage container 3 can flow out to the consumer, e.g. a patient. This process is only interrupted when the lower diode measuring device 7 detects that the lower filling level 9 has been reached as is indicated by 9 in FIG. 6. In this state, the lower squeezing element 14 is moved from its open position into its closed position. The process of filling the storage container 3 can then be begun again.

Thus, the system 1 enables several filling and discharge cycles of the storage container 3 to be effected over time under the control of the control unit ST whereby the time interval between two discharge cycles is freely selectable. The system 1 according to the invention is thus particularly suitable for enteral nutrition since once the desired parameters have been established, accurately dosed volumes of liquid nutrients can automatically be transferred and administered to a patient at defined time points over a desired length of time, e.g. one day, without further intervention in the system.

Although this is not illustrated, the actuating organs 10, 11 can be provided with suitable detecting means in order to transmit an alarm signal to the control unit ST and/or switch the system off in the event of a malfunction of an actuating organ. Furthermore, the system can be equipped with an additional battery serving as an emergency power source.

An embodiment of the storage container 3 having a ventilating device 17 is illustrated in FIG. 7. The storage container 3 is slightly conical in shape. A cap 15 is mountable on the storage container 3 and connected to the supply device 4.

As FIG. 7 shows furthermore, the storage container 3 can be in the form of a drip chamber. The ventilating device 17 is held on a projection 18 of the cap 15 by means of a retaining element 19.

Finally it should be noted that the system 1 according to the invention can be equipped with additional diode measuring units for the detection of further filling levels so that not just one volume is defined in accord with the lower and upper filling levels, but two or several partial volumes are defined in the storage container, said partial volumes being deliverable successively by appropriately controlling the squeezing elements 13, 14 by means of the control unit ST.

1. A system for metering and delivering a liquid medium, in particular for enteral nutrition in medical applications, including a storage container having a certain volumetric capacity and a supply device and a discharge device for the medium, whereby the supply and discharge of the medium into and out of the storage container is effected by the force of gravity, said system further including a detecting device for determining at least a lower and at least an upper filling level of the medium in the storage container and for outputting appropriate detection signals, and controllable actuating means for closing or opening the supply device or the discharge device, respectively, said detection signals output from the detecting device are supplied to a control unit for supplying setting signals to the controllable actuating organs responsive to a given program sequence in dependence on the detection signals.

2. The system according to claim 1, wherein said detecting device comprises at least one pair of diode measuring units spaced from each other in the direction of the gravitational force in correspondence with the upper and lower filling level.

3. The system according to claim 2, wherein said diode measuring device associated with the upper filling level is arranged in such a manner as to prevent scanning of the inflowing medium stream.

4. The system according to claim 1, wherein each said controllable actuating organ is movable into the closed or open position by a solenoid or a stepping motor.

5. The system according to claim 1, further including position detecting means for detecting the position of the controllable actuating organs.

6. The system according to claim 1, wherein said storage container is provided with a ventilating device.

7. The system according to claim 1, wherein said control unit is integrated into the system.

8. The system according to claim 1, wherein said detection signals output from the detecting device and the setting signals for the controllable actuating organs are applicable to an interface for connection to an external control unit.
